(19) United States
(12) Plant Patent Application Publication
(10) Pub. No.: US 2026/0372220 P1
(43) Pub. Date: Nov. 19, 2026

(54) Miniature Rose Plant Named 'Poulpar129'

(50) ;

(71) Applicant: Mogens Nyegaard Olesen, Fredensborg (DK)

(72) Inventor: Mogens Nyegaard Olesen, Fredensborg (DK)

(73) Assignee: Poulsen Roser A/S, Fredensborg (DK)

(21) Appl. No.: 18/831,804

(22) Filed: Sep. 15, 2025

(30) Foreign Application Priority Data
Sep. 19, 2024 (QZ) 20242191

(51) Int. Cl. A01H 6/74; A01H 5/02

(52) U.S. Cl. CPC A01H 6/749; A01H 5/02
USPC PLT/122

(57) ABSTRACT

A new garden rose plant of the Miniature class which has abundant, red flowers and attractive foliage. This new and distinct variety has shown to be uniform and stable in the resulting generations from asexual propagation.

Botanical designation:

Rosa hybrida.

Variety denomination:

‘Poulpar129’.

This application claims priority to Plant Breeder's Rights Application Number 2024/2191, which was filed at the Community Plant Variety Rights Office in the European Union on Sep. 19, 2024, the contents of which are hereby incorporated by reference for all purposes.

### SUMMARY OF THE INVENTION

The present invention constitutes a new and distinct variety of rose plant which originated from a controlled crossing between the female seed parent, an unnamed seedling, and the male pollen parent, also an unnamed seedling. Both of the parent varieties are non-patented.

The two parents were crossed during the summer of 2016 and the resulting seeds were planted in a controlled environment in Fredensborg, Denmark. The new variety, named ‘Poulpar129’, originated as a single seedling from the stated cross.

The new variety may be distinguished from its male pollen parent and female seed parent primarily by the following characteristics. The male pollen parent plant has a flower colour of Red Group 46B while the claimed plant has a flower colour of Red Group 53A. The female seed parent plant grows to a mature height of 40 cm, while ‘Poulpar129’ grows to a mature height of about 25 cm.

The objective of the hybridization of this rose variety was to create a new and distinct variety with unique qualities, such as:
1. Uniform and abundant red flowers with long shelf-life;
2. Vigorous, but compact growth when propagated on its own roots;
3. Suitability for commercial production in small containers;
4. Exceptional disease resistance.

This combination of qualities is not present in previously available commercial cultivars of this type, known to the inventor, and distinguish ‘Poulpar129’ from all other varieties of which we are aware.

As part of the rose development program, Mogens N. Olesen germinated the seeds from the aforementioned hybridization during winter of 2016 and conducted evaluations on the resulting seedlings in a controlled environment in Fredensborg, Denmark. ‘Poulpar129’ was selected in the spring of 2017 by the inventor as a single plant from the progeny of the aforementioned hybridization.

Asexual reproduction of ‘Poulpar129’ by rooted cuttings was first done by Mogens N. Olesen in the nursery in Fredensborg, Denmark in July 2017. This initial and other subsequent asexual propagations conducted in controlled environments have demonstrated that the characteristics of ‘Poulpar129’ are true to type and are transmitted from one generation to the next.

### DESCRIPTION OF THE DRAWING

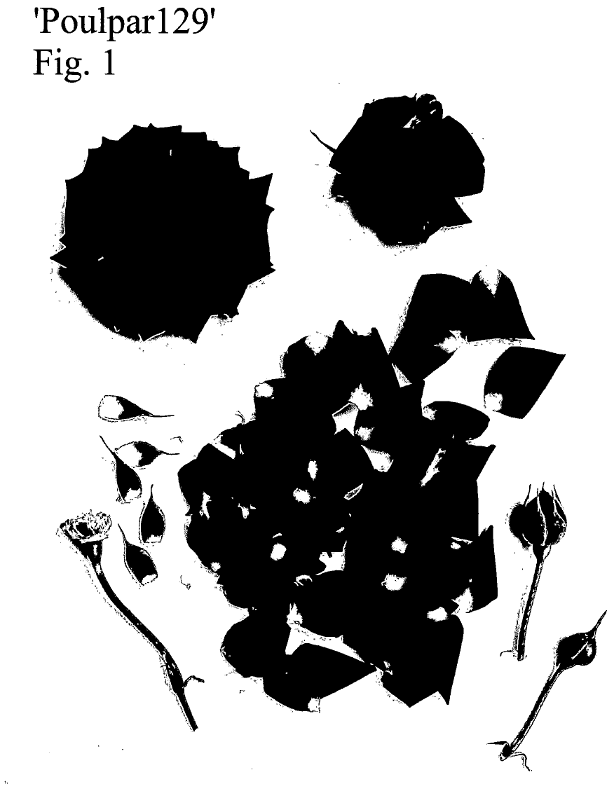

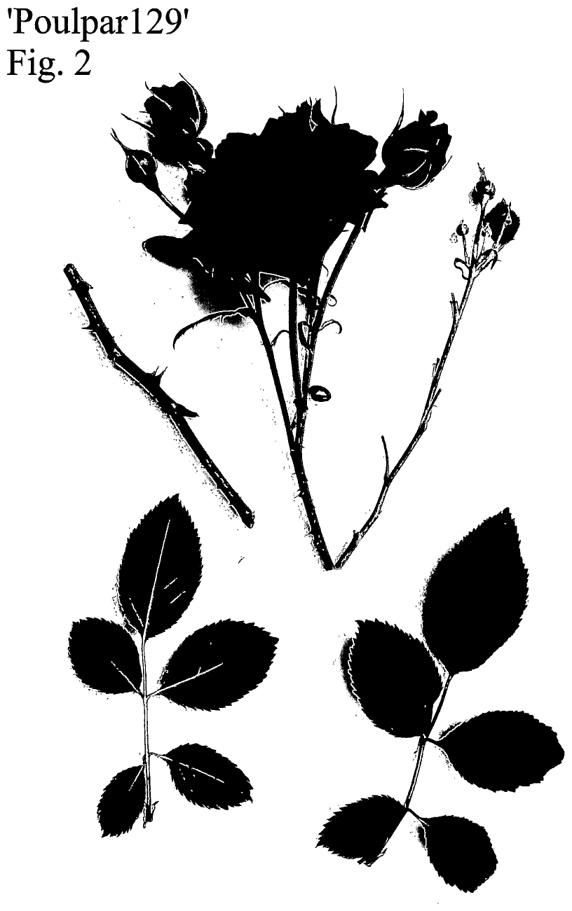

The accompanying color illustrations show as true as is reasonably possible to obtain in color photographs of this type, the typical characteristics of the buds, flowers, leaves, and stems, of ‘Poulpar129’. Specifically illustrated in FIG. 1 of the drawings are open flowers, at various stages of maturation, viewed from above, flower petals and sepals detached, revealing reproductive flower parts, and flower buds. Specifically illustrated in FIG. 2 of the drawings is a cluster of flowers quantifying the abundance of flowers exhibited on a single branch, bare stem exhibiting prickles, and mature leaves viewed from above and underneath. Plants shown are 3 months old.

### DETAILED DESCRIPTION OF THE VARIETY

The following is a description of ‘Poulpar129’, as observed in its growth in a glasshouse nursery in Odense Denmark. Observed plants are 3 months of age, and were grown on their own roots in 17 cm pots. Color references are made using the Royal Horticultural Society (London, England) Colour Chart, 2001, except where common terms of color are used.

For a comparison, several physical characteristics of the rose variety ‘Poulpar114’, U.S. Plant Pat. No. 32,297 are compared to the claimed plant. While ‘Poulpar129’ has 66 flower petals, ‘Poulpar114’ has 35 flower petals. The claimed plant produces flowers in clusters of 3 to 7 flower buds per stem, while ‘Poulpar114’ typically produces fewer flower buds on each stem, 3 on average. Open flowers of ‘Poulpar129’ are generally Red Group 53A in color while ‘Poulpar114’ has a general tonality of Red Group 46B.

### Flower and Flower Bud

Blooming habit: Continuous.
Flower bud:
   Size.—Upon opening, 19 mm in length from base of receptacle to end of bud. Bud diameter is 11 mm.
   Bud form.—Globose.
   Bud color.—As sepals divide petals are Red Group 53A.
   Sepal inner surface.—Color: Yellow-Green Group 146D. Surface: Lightly pubescent.
   Sepal outer surface.—Color: Yellow-Green Group 144A. Texture: Smooth.
   Sepal Shape.—Apex: Cirrhose. Base: Flat at union with receptacle.
   Sepal Margin.—Margins have no foliaceous appendages on three of the five sepals.
   Sepal size.—About 29 mm long and 9 mm wide.
   Receptacle.—Texture: Smooth. Size: 7 mm in height, 6 mm wide. Color: Yellow-Green Group 144A. Shape: Campanulate.
   Pedicel.—Surface: Rough with stipitate glands. Length: About 35 mm. Diameter: 2 mm on average. Color: Yellow-Green Group 144A. Strength: Moderate.
   Peduncle.—Length: 2 to 5 cm. Diameter: About 3 mm. Color: Yellow-Green Group 144A. Texture: Rough with small prickles.
Flower bud development: Flower buds are borne in clusters of 3 to 7 flower buds per stem.
Flower bloom:
   Fragrance.—Moderate rose scent.
   Duration.—The blooms have a duration on the plant of approximately 25 days. Petals fall cleanly away from plant after flowers have fully matured.
   Size.—Flower diameter is about 70 mm when open. Flower depth is 30 mm.
   Flower shape.—Double rosette, with many overlapping petals packed into quarter sections. From above star shaped.
   Shape of flower, side view.—The upper portion is convex. The lower portion is concave.
Petalage: Under normal conditions, flowers have about 66 petals.
General tonality of flower: Open flowers are Red Group 53A. After 10 days flower colour changes to Red Group 53A with light intonations of Red-Purple Group N57B.
   Petal Color.—Upon opening, outer petals Upper surface: Red Group 53C to Red Group 53C. Lower surface: Red-Purple Group 61C with occasional streaks of White Group 155A. Upon opening, inner petals: Upper surface: Red Group 53A. Occasionally streaks of Green Yellow Group 1D. Lower surface: Red-Purple Group 61C. Basal petal spots, upon opening: Upper surface: Yellow Group 8C. Lower surface: Yellow Group 8D. After opening, outer petals Upper surface: Red Group 53C to Red Group 53C with light intonations of Red-Purple Group N57B. Lower surface: Red-Purple Group 61C with occasional streaks of White Group 155A. After opening, inner petals: Upper surface: Red Group 53A. Occasionally streaks of Green Yellow Group 1D. Lower surface: Red-Purple Group 61C. Basal petal spots, after opening: Upper surface: Yellow Group 8C. Lower surface: Yellow Group 8D.
Petals.
   Petal Reflex.—Strong.
   Margin.—Entire and uniform. No undulations.
   Shape.—Broad and elliptic. Apex shape: Rounded. Base shape: Acute.
   Size.—40 mm (1)×36 mm (w). Texture: Smooth. Thickness: Average.
Petaloids:
   Size.—15 mm (l) by 8 mm (w).
   Quantity.—About 15.
   Shape.—Elliptical with an acute base and rounded apices.
   Color.—Red Group 53A on the upper surface with a basal petaloid spot of Yellow Group 8A. The lower surface is Red-Purple Group 61C with a basal petaloid spot of Yellow Group 8B.
Reproductive Flower Parts.
   Pollen.—None observed.
   Anthers.—. Size: 2 mm in length. Color: Yellow Group 11B. Quantity: 50 on average.
   Filaments.—. Color: Yellow Group 11A Length: 6 mm.
   Pistils.—Length: 7 mm. Quantity: 40 on average.
   Stigmas.—Color: Greyed-Yellow Group 162D.
   Styles.—Color: White Group 155A.
   Location of stigmas.—Even in location relative to the length of the filaments and the height of the anthers.
   Hips.—None Observed.
Plant
Plant growth: Upright. Plants are, on average, 25 cm in height, and 28 cm wide.
Stems.
   Color of juvenile growth.—Yellow-Green Group 144B.
   Color of mature growth.—Yellow-Green Group 144A.
   Length.—Canes are about 20 cm from the base of the plant to the flowering portion.
   Diameter: About 4 mm. Color of mature growth: Length: Diameter: Internodes: On mature canes about 25 mm between nodes. Surface Texture: Young wood: Smooth. Older wood: Rough with many small prickles.
   Long Prickles: Incidence: 9 prickles per 10 cm of stem. Size: Average length of prickles on mature stems is 8 mm. Shape: Upper portion is linear. Lower portion is concave. Color: Juvenile prickles: Greyed-Red Group 182A. Mature prickles: Greyed-Red Group 182B.
   Plant foliage.—Compound leaf: About 155 mm (1)×115 (w). Quantity: 3 leaves per 10 cm of stem on average. Leaf bearing angle to the stem: About 60 degrees. Color of juvenile foliage: Upper side: Yellow-Green Group 144A. Lower side: Yellow-Green Group 144B. Color of mature foliage: Upper side: Yellow-Green Group 147A. Lower side: Yellow-Green Group 147B.
Plant leaves and leaflets
   Stipules—. Size: 11 mm long, 4 mm wide. Quantity: 2 per compound leaf. Shape: Linear, slightly broad based with outward extending apices. Margins: Finely serrated. Color: Yellow-Green Group 144A.
   Petiole—. Length: About 27 mm. Diameter: 2 mm. Upper surface color: Yellow-Green Group 144A. Lower surface color: Yellow-Green Group 144A.
   Rachis—. Length: 55 mm on average. Upper surface color: Yellow-Green Group 144A. Lower surface color: Yellow-Green Group 144A. Texture: Rough with small Greyed-Yellow Group 160A prickles.
   Leaflet—. Quantity: Normally 5 leaflets. Margins: Serrated. Size: Terminal leaflets are about 75 mm long, 40 mm wide. Shape: Elliptical. Base: Rounded. Apex: Acute.
   Texture—. Smooth.
   Thickness—. Average.
   Arrangement—. Odd pinnate.
   Venation—. Reticulate.
   Glossiness—. Very glossy.
Disease resistance: Above average resistance to powdery mildew Sphaerotheca pannosa var. rosae, downy mildew Peronospora sparsa, rust Phragmidium spp., black spot Diplocarpon rosae, and Botrytis cinerea under normal growing conditions.
Cold hardiness: The variety is tolerant to USDA Cold Hardiness Zone 6.
Heat Tolerance:

The variety has been found to be suitable for climate conditions found in the American Horticulture Society heat zone 7.

I claim:

1. A new and distinct variety of rose plant named ‘Poulpar129’ substantially as described and illustrated herein.
